SEQUENCE LISTING (17 sequences)

SEQ ID NO: 1 - protein, 191 aa, Phragmatopoma californica
MKVFIVLALVSAAYGCGVGIGCAGGRCGGACGGKGYGYGGKLGYGAYGKGGIGGYGYGKGCVGGYGYGGLGAGKLGGYGYGGSKCGGYGYGGQKLGGYGYGGKKLGGYGYAAKKVGGYGYGAKKVGGYGYGAKKVGGYGYGAKKVGGYGYGAKKVGGYGYGAKKVGGYGYGAKKVGGYGYGVKKVGGYGYG

SEQ ID NO: 2 - protein, 210 aa, Phragmatopoma californica
MKVLIFLATVAAVYGCGGAGGWRSGSCGGRWGHPAVHKALGGYGGYGAHPAVHAAVHKALGGYGAGAYGAGAWGHPAVHKALGGYGAGAWGHPAVHKALGGYGGYGAHPAVHVAVHKALGGYGAGACGHKTGGYGGYGAHPVAVKAAYNHGFNYGANNAIKSTKRFGGYGAHPVVKKAFSRGLSHGAYAGSKAATGYGYGSGKAAGGYGY

SEQ ID NO: 3 - protein, 272 aa, Phragmatopoma californica
MPTLYKKVGKLVILAIIVTVASVASAGYPTYSPSGGTHSGYNGPHGNVVKKTYRGPYGAGAAKAWNGYHGAGYTSVHHGPASTSWHTSWSNKKGGYGYGLKNKGYGYGLKKVGYGVGLHAAGWHGVGPYGAGYHGAGWNGLGYHGAGYGVHGVGLHGAGYGLHGVGLHGVGYGLHGVGLHGAGYGLHGVGLHGAGYGIHGVGLHGAGYGIHGVGLHGVGYGLHGVGLHGAGYGLHGVGLHGAGYGIHGVGLHGAGCGIHKTACYGVGLHGHY

SEQ ID NO: 4 - protein, 144 aa, Phragmatopoma californica
MKFLVLLALVASASAYYPLMGGFHGGWHAPMVHGGLYHGGWHAPMVHGGLYHGGWHAPIVHGGWHAPVFHAPAPIHTVSHSVVNHVPMMPMWHHPAPAPAPAPRPGRTIILGGGKYGPFGKYGGGAGLLALGALGGNGGFWKRR

SEQ ID NO: 5 - protein, 328 aa, Phragmatopoma californica
MLFYNANFVQKSWVLILLGLAAVVACSEYDKGLGGYGRPSYGGRRGYGGRRGLQYHGKYQGRCEYDGLYFRDEKSFVYCSNRNSYIQPCAPGTRNSPYTKYNRGSKYNYRDFCEVNLVDSGYVPKPGYLPAPKKAYPTKVYDLKVDYAPKVDYAPKVDYAPKVDYAPKVDYVAPKASYVPPKASYVDPTPTYGYEAPFKGGYDKPSYGKDVDTSYESKTTYTVEKTADKGYGKGYGDKEISAKKSYTLTEKRDYDTGYDNSRSDEDSKEYGYDNDRSESYERTESYTDERTDGYGTQKVEYTQQSEYDRVTRRGIWLHKGTEVEHVLY

SEQ ID NO: 6 - protein, 136 aa, Phragmatopoma californica
  misc_feature (49)..(49): Xaa can be any naturally occurring amino acid
MNTFVVLAAIVAVAACSGGYDGRQYTYRGRYNNKCGNDGLYFKDDKNFXFCSNGNSYVQPCAPGTRNSGYNNYKQGSIYNYRDFCDVNLVDEGYGVGAKPGYNKGYNPGYNPGYGGYNPGYSTGYGGYKAGPGPYW

SEQ ID NO: 7 - protein, 158 aa, Phragmatopoma californica
MKLALLLLVAVCAAVAVNACGPLGCSGGYGGVLKCGVGGCALGGYGGGYSAGIGGYGIKRLGCRGGRCGLRRRVGCRGGRCGLRGRLGCRGGRCGLRKLGCRGGRCGLRGRLGCRGGRCGLRKRLGCRGGRCGRGGYGGGYGGVCSKGVCGGYPAYGK

SEQ ID NO: 8 - protein, 236 aa, Phragmatopoma californica
MKVSIAVLIMCCIAAVLADGYKSKNGGQAGGYGGYGSGYGGGYGGGYDGGYGGEKGKSGKGYGDRKGKSEKGYGNGKGKGGSGYGGGYDGGYGGGKGKSGSGYGGGYDGGYGGGKGKSGSGYGGGYDGGYDGGYGGGKGKSGSGFGGGYDGGYDGGYGGGKGKSGSGYGGGYDGGYDGGYGGGKGKSGSGYGGGYDGGYDGGYGGGKGKSGSGYGGGYDGGYDGRYGGGKGKSGSG

SEQ ID NO: 9 - protein, 191 aa, Phragmatopoma californica
MKLICLVLLAVCIVAVSASSSSGGRRRRVIVIGNRGRAPARPRSNIHYHMHAPQPRMMMAPRMMMAPMMMAPMAMPATSHVYQSVSYPGAMYRYGLGSLGGGFISGGLGGIVGGGLHGGVVTSGLHGGVVTSGLHGGVVTSGLHGGLVSGGWHSGVVSHGGLIGGGIHTTYGGFHKGVVHGGYTGHYGKRR

SEQ ID NO: 10 - protein, 102 aa, Phragmatopoma californica
MKLAVFALLVAFAIVYTAEGLVYGGQKGYGYGGKGYGYGCTGGYGLYGGKGYGYGKGYGYGCRGGYGYGKGYGYGGKYRGYGYGNKVGYGYGQQLGYKNGRK

SEQ ID NO: 11 - protein, 91 aa, Phragmatopoma californica
LDGGCKPTGGFIKGSVGPCGGYNHQHVVGPNGAHGRRVGYGPNGKYSQIYGNGPGGRYSHTVVYPRVRPYGGYGFKGGYGGYHGVGYKGGY

SEQ ID NO: 12 - protein, 145 aa, Phragmatopoma californica
MKVFVAALLLCCIAAAAAEDGYGFGYDGYGSGYGYDGYSYGGDKGYGYGKGKGYGYEGGKGYGYEGGKGYGHEEGKGYGHEGGKGYGYEGGKGYGYGGGKGYGHDGGKGYGHDGGKGYGYGGGKGYGHEGGKGYGYEGGKGYGKY

SEQ ID NO: 13 - protein, 134 aa, Phragmatopoma californica
MRIVICLLVLVAGAYGIGCGYGGYGGYGGGFHGGYIGYHGGYPGYSGGFRGYGYPGRVHTNVVHHNIPVFMPPPMPRRAPAPAPRGRTIILGGGKYGLFGKKSKNKGFGGLGVLSLLGGLGGKGGGGIRFLGRK

SEQ ID NO: 14 - protein, 226 aa, Phragmatopoma californica
MKVIILLATVAAVYGCGGWNGGFGGGKACGGGWGAKALGGYGSYNGNGYGAHPVAVKSAFNKGVSYGARSAVKATRGFAYGKGSSYGYGAHPAVKSAFGNGFKTGAHAAVNGYGYGAVKSALSGGYGYGSYGAHPAVKSAYRKGLSYGAKSAVKATRGFAYGRSGYGAHPVVKSAFSNGFKYGAHAAVKATNGYGYGAVHPAVKAAVKGGYGYGNKGGYGAGYAAY

SEQ ID NO: 15 - protein, 89 aa, Phragmatopoma californica
MKVFVATLLLCCIAAAAAAGYGNGYAGYGSGYAGYGTGYAGYGSGYGYDGYGYGGGKGYGYGGDKGYGYGGKGYGYGGQKGYGYGYGKY

SEQ ID NO: 16 - protein, 105 aa, Phragmatopoma californica
MKLLLLFALAAVAVALPYGYSGKPGYGYDAVDAVYNRLADKQQAVNRKAEYVGAGTGTAKYNGVPGANYGYENDRKYGYDNKGYGGYGDKGYGGYGDKGLYDGYY

SEQ ID NO: 17 - protein, 275 aa, Phragmatopoma californica
KYYDDEKRDADKYRKPSYNPYNTYKDYPPKKIYNDDEKRDADQYRISYNPYNTYKDYPPKKKYYDDEKRDAYKYRNPSYNPYNTYKDYPPKKIYYDDEKRDADQYRNPSYNPYNTYKDYPPKKKYYDDEKRDADQYRNPSYNPYNTYKDYLPKKKYYDDEKRDADQYRKPSYNPYNSYKDYPPKKKYYDDEKRDADQYRNPSYNPYNTYKDYLPKKKYYDDEKRDADQYRNPSYNPYNTYKDYPPKKKYYDDEKRDADQYRNPSYNPYNTYKDYP

(19) United States
(12) Patent Application Publication
(10) Pub. No.: US 2014/0220082 A1
(43) Pub. Date: Aug. 7, 2014

(54) ADHESIVE COMPLEX COACERVATES AND METHODS OF MAKING AND USING THEREOF

(71) Applicant: Russell J. Stewart, Salt Lake City, UT (US)

(72) Inventor: Russell J. Stewart, Salt Lake City, UT (US)

(73) Assignee: UNIVERSITY OF UTAH RESEARCH FOUNDATION, Salt Lake City, UT (US)

(21) Appl. No.: 14/128,656

(22) PCT Filed: Jun. 27, 2012
(86) PCT No.: PCT/US12/44299

§ 371 Date: Mar. 27, 2014

Related U.S. Application Data
(60) Provisional application No. 61/501,863, filed on Jun. 28, 2011.

(51) Int. Cl. A61K 47/32; A61L 24/06

(52) U.S. Cl. CPC A61K 47/32; A61L 24/06
USPC 424/400; 525/287; 514/772.1

(57) ABSTRACT

Described herein is the synthesis of adhesive complex coacervates and their use thereof. The adhesive complex coacervates are produced by reacting (a) at least one poly anion comprising a plurality of activated ester groups, and (b) at least one polycation comprising a plurality of nucleophilic groups, wherein the nucleophilic groups react with the activated ester groups to produce a new oovalent bond between the polycation and the polyanion. The adhesive complex coacervates described herein have low interfacial tension with water and wettable substrates. When applied to a wet substrate they spread over the interface rather than beading up. The adhesive complex coacervates have numerous biological applications as bioadhesives and drug delivery devices. In particular, the adhesive complex coacervates described herein are particularly useful in wet or underwater applications and situations where water is present such as, for example, physiological conditions.

### CROSS REFERENCE TO RELATED APPLICATIONS

This application claims priority upon U.S. provisional application Ser. No. 61/501,863, filed Jun. 28, 2011. This application is hereby incorporated by reference in its entirety.

### ACKNOWLEDGEMENTS

The research leading to this invention was funded in part by the National Institutes of Health, Grant No. R01 EB006463 and the Office of Naval Research, Grant No. N000141010108. The U.S. Government has certain rights in this invention.

### CROSS REFERENCE TO SEQUENCE LISTING

Proteins described herein are referred to by a sequence identifier number (SEQ ID NO). The SEQ ID NO corresponds numerically to the sequence identifiers <400>1, <400>2, etc. The Sequence Listing, in written computer readable format (CFR), is incorporated by reference in its entirety.

### SUMMARY

Described herein is the synthesis of adhesive complex coacervates and their use thereof. The adhesive complex coacervates are produced by reacting (a) at least one polyanion comprising a plurality of activated ester groups, and (b) at least one polycation comprising a plurality of nucleophilic groups, wherein the nucleophilic groups react with the activated ester groups to produce a new covalent bond between the polycation and the polyanion. The adhesive complex coacervates have several desirable features when compared to conventional adhesives. The adhesive complex coacervates are effective in wet applications. The adhesive complex coacervates described herein have low interfacial tension with water and wettable substrates. When applied to a wet substrate they spread over the interface rather than beading up. The adhesive complex coacervates have numerous biological applications as bioadhesives and drug delivery devices. In particular, the adhesive complex coacervates described herein are particularly useful in underwater applications and situations where water is present such as, for example, physiological conditions.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the formation of complex coacervates by adjusting the pH of a solution of polycations and polyanions. (A) Oppositely charged polycations and polyanions associate into colloidal polyelectrolyte complexes (PECs) at a pH (˜6 for the example shown) where the PECs have a net positive charge as represented in (E). (B) By raising the pH (to ˜7 for the example shown), the net charge on the colloidal PECs approaches net charge neutrality where upon the complexes associate and separate as a dense fluid phase, i.e., a complex coacervate. (C) The complex coacervate has several ideal properties as the basis of underwater adhesives: density greater than water so they sink rather than float, water immiscibility that prevents mixing in a watery environment, and injectability allowing convenient application onto wet surfaces or underwater. (D) The complex coacervates can readily be spread on wet hydrophilic substrates because of the low interfacial tension with water and wettable surfaces.

FIG. 2 shows a four-arm polyaminoacrylamide useful as a polycation herein.

FIG. 3 shows a polyphosphate-co-carboxylate useful as a polyanion that can subsequently be converted to an activated ester.

FIG. 4 shows the incorporation of reinforcing components into the adhesive complex coacervates to improve mechanical properties. (Left) Water-soluble, or water-suspendable components, or solid particles present in the solution before the complex coacervate condenses will be entrapped in the watery phase of complex coacervate network (right).

FIG. 5 shows a reaction scheme for crosslinking a polycation and polyanion in a coacervate with ethylenediamine carbodiimide (EDC).

FIG. 6 shows bond strength measurements for coacervates crosslinked with varying ratios of EDC/carboxylate groups present on the polyanion.

FIG. 7 shows rheological measurements for coacervates crosslinked with varying ratios of EDC/carboxylate groups present on the polyanion over time.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, a palmitate group. A “lower alkyl” group is an alkyl group containing from one to six carbon atoms.

The term “aryl group” as used herein is any carbon-based aromatic group including, but not limited to, benzene, naphthalene, etc. The term “aryl group” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “activated ester group” as used herein is any carboxyl group that has been converted to an ester group that readily reacts with a nucleophilic group to produce a new covalent bond. Examples of activated ester groups are provided below.

The term “nucleophilic group” includes any groups capable of reacting with an activated ester. Examples include amino groups, thiols groups, hydroxyl groups, and their corresponding anions.

The term “carboxyl group” includes a carboxylic acid and the corresponding salt thereof.

The term “amino group” as used herein is represented as the formula —NHRR″, where R and R′ can be any organic group including alkyl, aryl, carbonyl, and the like.

Described herein are adhesive complex coacervates and their applications thereof. In general, the complex coacervates are a mixture of polycations and polyanions in balanced proportions to produce a phase separated fluid at a desired pH. In one aspect, the adhesive complex coacervates are produced by the process comprising reacting (a) at least one polyanion comprising a plurality of activated ester groups, and (b) at least one polycation comprising a plurality of nucleophilic groups, wherein the nucleophilic groups react with the activated ester groups to produce a new covalent bond between the polycation and the polyanion.

The adhesive complex coacervate is an associative liquid with a dynamic structure in which the individual polymer components can diffuse throughout the entire phase. As described above, the adhesive complex coacervates exhibit low interfacial tension with water and hydrophilic substrates. In other words, when applied to substrates either under water or that are wet the complex coacervate spreads evenly over the interface rather than beading up. The coacervates can also penetrate cracks and defects. Additionally, upon intermolecular crosslinking (discussed in detail below), the adhesive complex coacervate forms a strong, insoluble, cohesive material. Conversely, polyeletrolyte complexes (PECs), which can be a precursor to the adhesive complex coacervates described herein, are small colloidal particles.

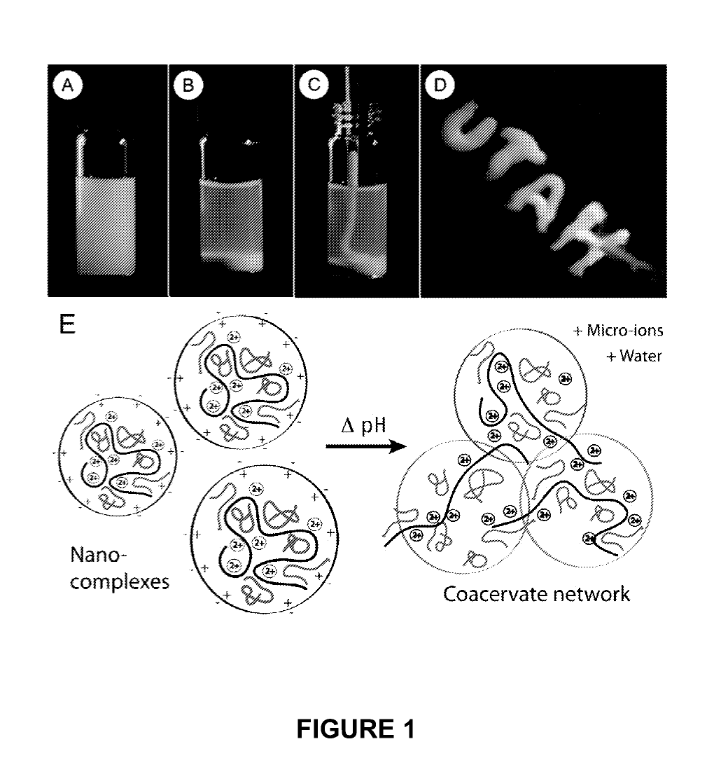

An exemplary model of the differences in phase behavior between the polyelectrolyte complexes (PEC) and the adhesive complex coacervate is presented in FIG. 1. At low pH the oppositely charged polyelectrolytes associate electrostatically into nano-complexes with a net positive surface charge that stabilizes the suspension (FIG. 1A). With increasing pH the net charge of the complexes approaches net neutrality (FIG. 1B). Thus, in certain aspects, the conversion of the PEC to complex coacervate can be “triggered” by adjusting the pH and/or the concentration of the multivalent cation. For example, the PECs can be produced at a pH of less than or equal to 4, and the pH of the PECs can be raised to greater than or equal to 7.0, from 7.0 to 9.0, or from 8.0 to 9.0 to convert the PECs to a complex coacervate. Alternatively, a solution of polycation can be mixed with a solution of polyanion such that when the two solutions are mixed the final pH of the mixture is conducive to the formation of the complex coacervate. In this embodiment, the concentration of the polycation and polyanion can be adjusted accordingly in order to produce a complex coacervate.

Each component used to prepare the adhesive complex coacervates and methods for making and using the same are described below.

### I. Polycations

The polycation is generally composed of a polymer backbone with a plurality of cationic groups at a particular pH. The cationic groups can be pendant to the polymer backbone and/or incorporated within the polymer backbone. In certain aspects, (e.g., biomedical applications), the polycation is any biocompatible polymer possessing cationic groups or groups that can be readily converted to cationic groups by adjusting the pH. In one aspect, the polycation is a polyamine compound. The amino groups of the polyamine can be branched or part of the polymer backbone. The amino group can be a primary, secondary, or tertiary amino group that can be protonated to produce a cationic ammonium group at a selected pH. In general, the polyamine is a polymer with a large excess of positive charges relative to negative charges at the relevant pH, as reflected in its isoelectric point (pI), which is the pH at which the polymer has a net neutral charge. The number of amino groups present on the polycation ultimately determines the charge of the polycation at a particular pH. For example, the polycation can have from 10 to 90 mole %, 10 to 80 mole %, 10 to 70 mole %, 10 to 60 mole %, 10 to 50 mole %, 10 to 40 mole %, 10 to 30 mole %, or to 20 mole % amino groups. In one aspect, the polyamine has an excess positive charge at a pH of about 7, with a pI significantly greater than 7. As will be discussed below, additional amino groups can be incorporated into the polymer in order to increase the pI value.

In one aspect, the amino group can be derived from a residue of lysine, histidine, or arginine attached to the polycation. Any anionic counterions can be used in association with the cationic polymers. The counterions should be physically and chemically compatible with the essential components of the composition and do not otherwise unduly impair product performance, stability or aesthetics. Non-limiting examples of such counterions include halides (e.g., chloride, fluoride, bromide, iodide), sulfate and methylsulfate.

In one aspect, the polycation can be a positively-charged protein produced from a natural organism. For example, a recombinant P. californica protein can be used as the polycation. In one aspect, Pc1, Pc2, Pc4-Pc18 (SEQ ID NOS 1-17) can be used as the polycation. The type and number of amino acids present in the protein can vary in order to achieve the desired solution properties. For example, Pct is enriched with lysine (13.5 mole %) while Pc4 and Pc5 are enriched with histidine (12.6 and 11.3 mole %, respectively).

In another aspect, the polycation is a recombinant protein produced by artificial expression of a gene or a modified gene or a composite gene containing parts from several genes in a heterologous host such as, for example, bacteria, yeast, cows, goats, tobacco, and the like.

In another aspect, the polycation can be a biodegradable polyamine. The biodegradable polyamine can be a synthetic polymer or naturally-occurring polymer. The mechanism by which the polyamine can degrade will vary depending upon the polyamine that is used. In the case of natural polymers, they are biodegradable because there are enzymes that can hydrolyze the polymers and break the polymer chain. For example, proteases can hydrolyze natural proteins like gelatin. In the case of synthetic biodegradable polyamines, they also possess chemically labile bonds. For example, β-aminoesters have hydrolyzable ester groups. In addition to the nature of the polyamine, other considerations such as the molecular weight of the polyamine and crosslink density of the adhesive can be varied in order to modify the degree of biodegradability.

In one aspect, the biodegradable polyamine includes a polysaccharide, a protein, or a synthetic polyamine. Polysaccharides bearing one or more amino groups can be used herein. In one aspect, the polysaccharide is a natural polysaccharide such as chitosan or chemically modified chitosan. Similarly, the protein can be a synthetic or naturally-occurring compound. In another aspect, the biodegradable polyamine is a synthetic polyamine such as poly(β-aminoesters), polyester amines, poly(disulfide amines), mixed poly(ester and amide amines), and peptide crosslinked polyamines.

In the case when the polycation is a synthetic polymer, a variety of different polymers can be used; however, in certain applications such as, for example, biomedical applications, it is desirable that the polymer be biocompatible and non-toxic to cells and tissue. In one aspect, the biodegradable polyamine can be an amine-modified natural polymer. For example, the amine-modified natural polymer can be gelatin modified with one or more alkylamino groups, heteroaryl groups, or an aromatic group substituted with one or more amino groups. Examples of alkylamino groups are depicted in Formulae IV-VI

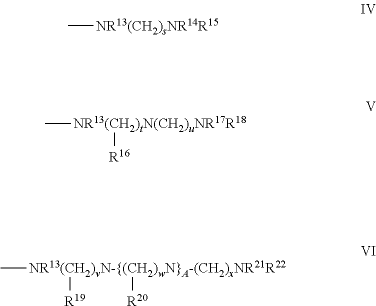

wherein R¹³-R²² are, independently, hydrogen, an alkyl group, or a nitrogen containing substituent;
s, t, u, v, w, and x are an integer from 1 to 10; and
A is an integer from 1 to 50,
where the alkylamino group is covalently attached to the natural polymer. In one aspect, if the natural polymer has a carboxyl group (e.g., acid or ester), the carboxyl group can be reacted with an alkyldiamino compound to produce an amide bond and incorporate the alkylamino group into the polymer. Thus, referring to formulae IV-VI, the amino group NR¹³ is covalently attached to the carbonyl group of the natural polymer.

As shown in formula IV-VI, the number of amino groups can vary. In one aspect, the alkylamino group is —NHCH₂NH₂, —NHCH₂CH₂NH₂, —NHCH₂CH₂CH₂NH₂, —NHCH₂CH₂CH₂CH₂NH₂, —NHCH₂CH₂CH₂CH₂CH₂NH₂, —NHCH₂NHCH₂CH₂CH₂NH₂, —NHCH₂CH₂NHCH₂CH₂CH₂NH₂, —NHCH₂CH₂CH₂NHCH₂CH₂CH₂CH₂NHCH₂CH₂CH₂NH₂, —NHCH₂CH₂NHCH₂CH₂CH₂CH₂NH₂, —NHCH₂CH₂NHCH₂CH₂CH₂NHCH₂CH₂CH₂NH₂, or —NHCH₂CH₂NH(CH₂CH₂NH)_{d}CH₂CH₂NH₂, where d is from 0 to 50.

In one aspect, the amine-modified natural polymer can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

In another aspect, the polycation can be a polycationic micelle or mixed micelle formed with cationic surfactants. The cationic surfactant can be mixed with nonionic surfactants to create micelles with variable charge ratios. The micelles are polycationic by virtue of the hydrophobic interactions that form a polyvalent micelle. In one aspect, the micelles have a plurality of amino groups capable of reacting with the activated ester groups present on the polyanion.

Examples of nonionic surfactants include the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15-S series from Union Carbide and Brij™ surfactants from ICI. Tergitol™ 15-S Surfactants include C₁₁-C₁₅ secondary alcohol polyethyleneglycol ethers. Brij™97 surfactant is polyoxyethylene(10) oleyl ether; Brij™58 surfactant is polyoxyethylene(20) cetyl ether; and Brij™76 surfactant is polyoxyethylene(10) stearyl ether.

Another useful class of nonionic surfactants include the polyethylene oxide condensates of one mole of alkyl phenol containing from about 6 to 12 carbon atoms in a straight or branched chain configuration, with ethylene oxide. Examples of nonreactive nonionic surfactants are the Igepal™ CO and CA series from Rhone-Poulenc. Igepal™ CO surfactants include nonylphenoxy poly(ethyleneoxy)ethanols. Igepal™ CA surfactants include octylphenoxy poly(ethyleneoxy)ethanols.

Another useful class of hydrocarbon nonionic surfactants include block copolymers of ethylene oxide and propylene oxide or butylene oxide. Examples of such nonionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers.

In other aspects, the nonionic surfactants include sorbitan fatty acid esters, polyoxyethylene sorbitan fatty acid esters and polyoxyethylene stearates. Examples of such fatty acid ester nonionic surfactants are the Span™, Tween™, and Myj™ surfactants from ICI. Span™ surfactants include C₁₂-C₁₈ sorbitan monoesters. Tween™ surfactants include poly(ethylene oxide) C₁₂-C₁₈ sorbitan monoesters. Myj™ surfactants include poly(ethylene oxide) stearates.

In one aspect, the nonionic surfactant can include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

Examples of cationic surfactants useful for making cationic micelles include alkylamine salts and quaternary ammonium salts. Non-limiting examples of cationic surfactants include: the quaternary ammonium surfactants, which can have up to 26 carbon atoms include: alkoxylate quaternary ammonium (AQA) surfactants as discussed in U.S. Pat. No. 6,136,769; dimethyl hydroxyethyl quaternary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

In one aspect, the polycation includes a polyacrylate having one or more pendant amino groups. For example, the backbone of the polycation can be derived from the polymerization of acrylate monomers including, but not limited to, acrylates, methacrylates, acrylamides, and the like. In one aspect, the polycation backbone is derived from polyacrylamide. In other aspects, the polycation is a block co-polymer, where segments or portions of the co-polymer possess cationic groups or neutral groups depending upon the selection of the monomers used to produce the co-polymer.

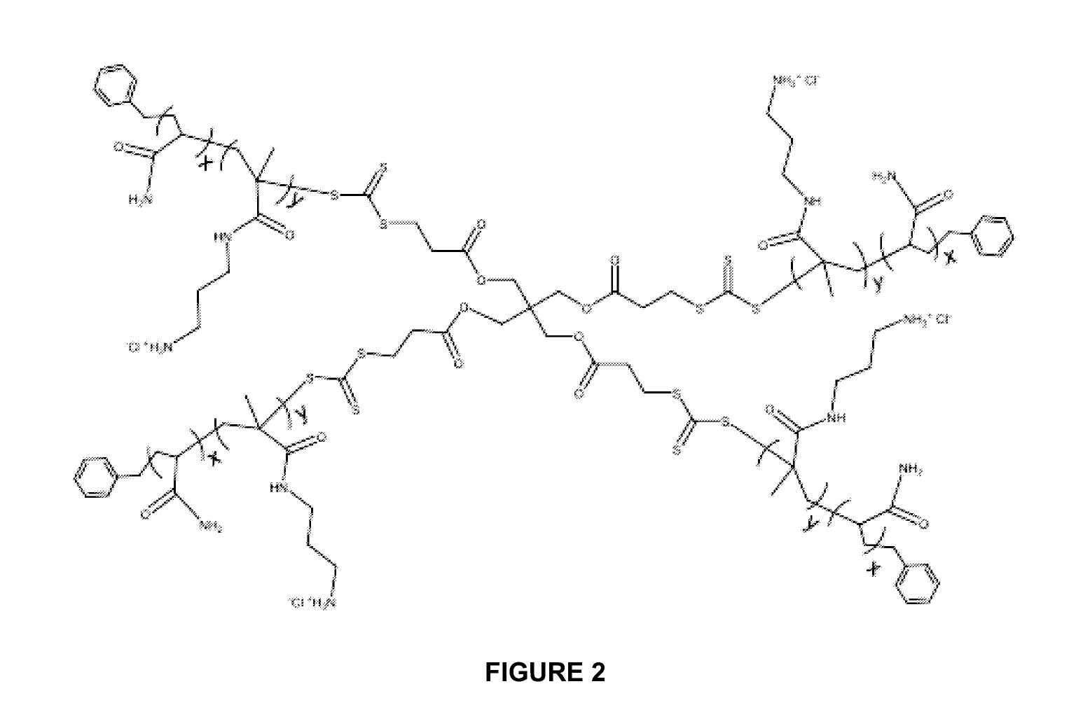

In other aspects, the polycation can be a dendrimer. The dendrimer can be a branched polymer, a multi-armed polymer, a star polymer, and the like. In one aspect, the dendrimer is a polyalkylimine dendrimer, a mixed amino/ether dendrimer, a mixed amino/amide dendrimer, or an amino acid dendrimer. In another aspect, the dendrimer is poly(amidoamine), or PAMAM. In one aspect, the dendrimer has 3 to 20 arms, wherein each arm comprises an amino group. FIG. 2 depicts an example of a branched polyamine. In this aspect, the polyamine has four arms with pendant free amino groups.

In one aspect, the polycation is a polyamino compound. In another aspect, the polyamino compound has 10 to 90 mole % primary amino groups. In a further aspect, the polycation polymer has at least one fragment of the formula I

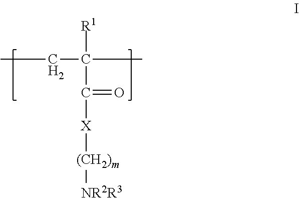

wherein R¹, R², and R³ are, independently, hydrogen or an alkyl group, X is oxygen or NR⁵, where R⁵ is hydrogen or an alkyl group, and m is from 1 to 10, or the pharmaceutically-acceptable salt thereof. In another aspect, R¹, R², and R³ are methyl and m is 2. Referring to formula I, the polymer backbone is composed of CH₂—CR¹ units with pendant —C(O)X(CH₂)ₘNR²R³ units. In one aspect, the polycation is the free radical polymerization product of a cationic primary amine monomer (3-amino-propyl methacrylate) and acrylamide, where the molecular weight is from 10 to 200 kd and possesses primary monomer concentrations from 5 to 90 mol %.

### II. Polyanions

Similar to the polycation, the polyanion can be a synthetic polymer or naturally-occurring. Examples of other naturally-occurring polyanions include glycosaminoglycans such as condroitin sulfate, heparin, heparin sulfate, dermatan sulfate, keratin sulfate, and hyaluronic acid. In these aspects, the glycosaminoglycan has pendant carboxylic acid groups that can be converted to activated ester groups. In other aspects, the polyanion can be a polysaccharide that can be chemically modified in order to incorporate of plurality of activated ester groups into the polysaccharide. In other aspects, acidic proteins having a net negative charge at neutral pH or proteins with a low pI can be used as naturally-occurring polyanions described herein. The anionic groups can be pendant to the polymer backbone and/or incorporated in the polymer backbone.

When the polyanion is a synthetic polymer, it is generally any polymer possessing anionic groups or groups that can be readily converted to anionic groups by adjusting the pH. Examples of groups that can be converted to anionic groups include, but are not limited to, carboxylate, sulfonate, boronate, sulfate, borate, phosphonate, or phosphate. Any cationic counterions can be used in association with the anionic polymers if the considerations discussed above are met.

In one aspect, the polyanion is a polyphosphate. In another aspect, the polyanion is a polyphosphate compound having from 5 to 90 mole % phosphate groups. For example, the polyphosphate can be a naturally-occurring compound such as, for example, highly phosphorylated proteins like phosvitin (an egg protein), dentin (a natural tooth phosphoprotein), casein (a phosphorylated milk protein), or bone proteins (e.g. osteopontin).

Alternatively, the polyphosphoserine can be a synthetic polypeptide made by polymerizing the amino acid serine and then chemically phosphorylating the polypeptide. In another aspect, the polyphosphoserine can be produced by the polymerization of phosphoserine. In one aspect, the polyphosphate can be produced by chemically or enzymatically phosphorylating a protein (e.g., natural serine- or threonine-rich proteins). In a further aspect, the polyphosphate can be produced by chemically phosphorylating a polyalcohol including, but not limited to, polysaccharides such as cellulose or dextran.

In another aspect, the polyphosphate can be a synthetic compound. For example, the polyphosphate can be a polymer with pendant phosphate groups attached to the polymer backbone and/or present in the polymer backbone. (e.g., a phosphodiester backbone).

In another aspect, the polyanion can be a micelle or mixed micelle formed with anionic surfactants, where the micelle has a plurality of activated ester groups. The anionic surfactant can be mixed with any of the nonionic surfactants described above to create micelles with variable charge ratios. The micelles are polyanionic by virtue of the hydrophobic interactions that form a polyvalent micelle.

Other useful anionic surfactants include, but are not limited to, alkali metal and (alkyl)ammonium salts of: 1) alkyl sulfates and sulfonates such as sodium dodecyl sulfate, sodium 2-ethylhexyl sulfate, and potassium dodecanesulfonate; 2) sulfates of polyethoxylated derivatives of straight or branched chain aliphatic alcohols and carboxylic acids; 3) alkylbenzene or alkylnaphthalene sulfonates and sulfates such as sodium laurylbenzene-4-sulfonate and ethoxylated and polyethoxylated alkyl and aralkyl alcohol carboxylates; 5) glycinates such as alkyl sarcosinates and alkyl glycinates; 6) sulfosuccinates including dialkyl sulfosuccinates; 7) isothionate derivatives; 8) N-acyltaurine derivatives such as sodium N methyl-N-oleyltaurate); 9) amine oxides including alkyl and alkylamidoalkyldialkylamine oxides; and 10) alkyl phosphate mono or di-esters such as ethoxylated dodecyl alcohol phosphate ester, sodium salt.

Representative commercial examples of suitable anionic sulfonate surfactants include, for example, sodium lauryl sulfate, available as TEXAPON™ L-100 from Henkel Inc., Wilmington, Del., or as POLYSTEP™ B-3 from Stepan Chemical Co, Northfield, Ill.; sodium 25 lauryl ether sulfate, available as POLYSTEP™ B-12 from Stepan Chemical Co., Northfield, Ill.; ammonium lauryl sulfate, available as STANDAPOL™ A from Henkel Inc., Wilmington, Del.; and sodium dodecyl benzene sulfonate, available as SIPONATE™ DS-10 from Rhone-Poulenc, Inc., Cranberry, N.J., dialkyl sulfosuccinates, having the tradename AEROSOL™ OT, commercially available from Cytec Industries, West Paterson, N.J.; sodium methyl taurate (available under the trade designation NIKKOL™ CMT30 from Nikko Chemicals Co., Tokyo, Japan); secondary alkane sulfonates such as Hostapur™ SAS which is a Sodium (C14-C17) secondary alkane sulfonates (alpha-olefin sulfonates) available from Clariant Corp., Charlotte, N.C.; methyl-2-sulfoalkyl esters such as sodium methyl-2-sulfo(C12-16)ester and disodium 2-sulfo(C12-C16) fatty acid available from Stepan Company under the trade designation ALPHASTE™ PC48; alkylsulfoacetates and alkylsulfosuccinates available as sodium laurylsulfoacetate (under the trade designation LANTHANOL™ LAL) and disodiumlaurethsulfosuccinate (STEPANMILD™ SL3), both from Stepan Company; alkylsulfates such as ammoniumlauryl sulfate commercially available under the trade designation STEPANOL™ AM from Stepan Company, and or dodecylbenzenesulfonic acid sold under BIO-SOFT® AS-100 from Stepan Chemical Co. In one aspect, the surfactant can be a disodium alpha olefin sulfonate, which contains a mixture of C₁₂ to C₁₆ sulfonates. In one aspect, CALSOFT™ AOS-40 manufactured by Pilot Corp. can be used herein as the surfactant. In another aspect, the surfactant is DOWFAX 2A1 or 2G manufactured by Dow Chemical, which are alkyl diphenyl oxide disulfonates.

Representative commercial examples of suitable anionic phosphate surfactants include a mixture of mono-, di- and tri-(alkyltetraglycolether)-o-phosphoric acid esters generally referred to as trilaureth-4-phosphate commercially available under the trade designation HOSTAPHAT™ 340KL from Clariant Corp., as well as PPG-5 cetyl 10 phosphate available under the trade designation CRODAPHOS™ SG from Croda Inc., Parsipanny, N.J.

Representative commercial examples of suitable anionic amine oxide surfactants those commercially available under the trade designations AMMONYX™ LO, LMDO, and CO, which are lauryldimethylamine oxide, laurylamidopropyldimethylamine oxide, and cetyl amine oxide, all from Stepan Company.

In one aspect, the polyanion includes a polyacrylate having one or more pendant phosphate groups. For example, the polyanion can be derived from the polymerization of acrylate monomers including, but not limited to, acrylates, methacrylates, and the like. In other aspects, the polyanion is a block co-polymer, where segments or portions of the co-polymer possess anionic groups and neutral groups depending upon the selection of the monomers used to produce the co-polymer.

In one aspect, the polyanion includes (1) two or more sulfate, sulfonate, borate, boronate, phosphonate, or phosphate groups and (2) a plurality of activated ester groups. In another aspect, the polyanion is a polyphosphate having a plurality of activated ester groups.

In another aspect, the polyanion is a polymer having at least one fragment having the formula X

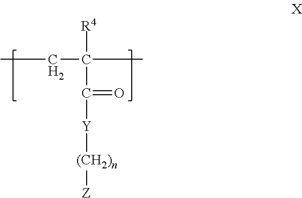

wherein R⁴ is hydrogen or an alkyl group;
n is from 1 to 10;
Y is oxygen, sulfur, or NR³⁰, wherein R³⁰ is hydrogen, an alkyl group, or an aryl group;
Z is an activated ester group,
or the pharmaceutically-acceptable salt thereof,
and at least one fragment having the formula XI

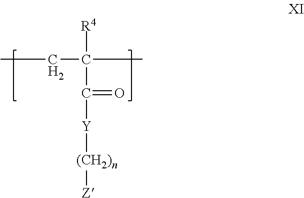

wherein R⁴ is hydrogen or an alkyl group;
n is from 1 to 10;
Y is oxygen, sulfur, or NR³⁰, wherein R³⁰ is hydrogen, an alkyl group, or an aryl group;
Z′ is an anionic group or a group that can be converted to an anionic group, or the pharmaceutically-acceptable salt thereof.

In one aspect, Z′ in formula XI is sulfate, sulfonate, borate, boronate, a substituted or unsubstituted phosphate, or a phosphonate. In another aspect, Z′ in formula XI is sulfate, sulfonate, borate, boronate, a substituted or unsubstituted phosphate, or a phosphonate, and n in both formulae X and XI is 2. In another aspect, the polyphosphate is the copolymerization product between (1) a phosphate acrylate and/or phosphate methacrylate and (2) a second acrylate and/or second methacrylate comprising a pendant activated ester groups covalently bonded to the second acrylate or second methacrylate.

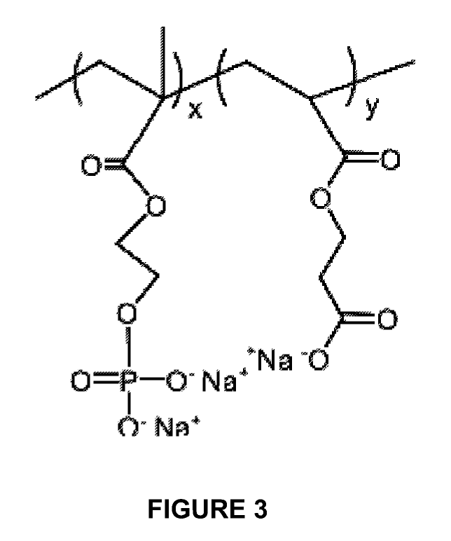

Also described herein are precursors to the polyanions described herein having a plurality of activated ester groups. For example, described herein are any of the polyanions described above where there is a plurality of carboxyl groups present on the polyanion prior to the conversion of the carboxyl groups to activated ester groups. FIG. 3 shows an example of a polyanion useful herein having pendant phosphate and carboxylate groups.

### III. Reinforcing Components

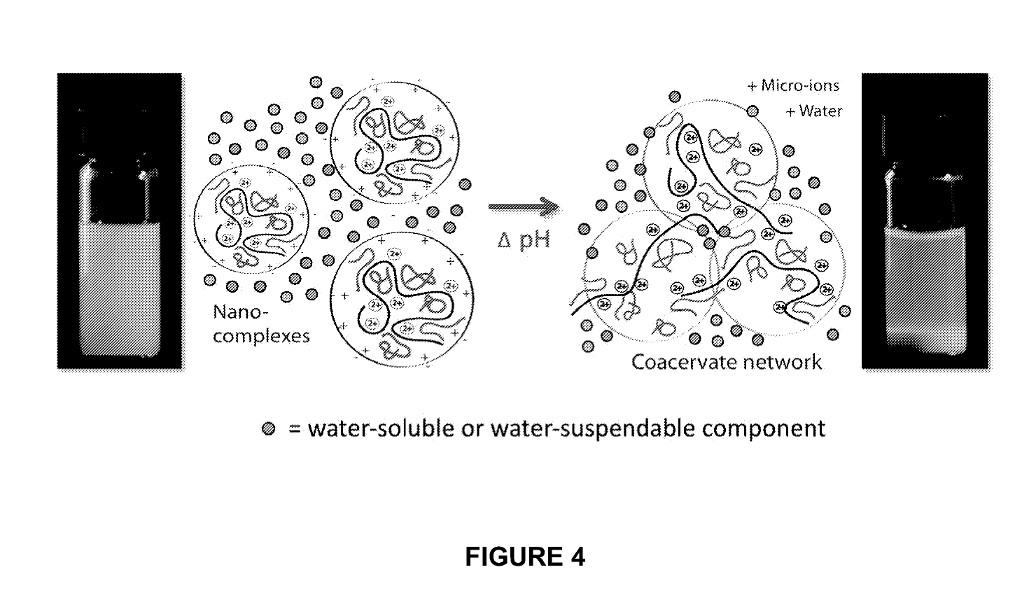

The coacervates described herein can optionally include a reinforcing component. The term “reinforcing component” is defined herein as any component that enhances or improves the mechanical properties (e.g., cohesiveness, fracture toughness, elastic modulus, the ability to release and bioactive agents, dimensional stability after curing, etc.) of the adhesive complex coacervate prior to or after the curing of the coacervate when compared to the same coacervate that does not include the reinforcing component. The mode in which the reinforcing component can enhance the mechanical properties of the coacervate can vary, and will depend upon the intended application of the adhesives as well as the selection of the polycation, polyanion, and reinforcing component. For example, upon curing the coacervate, the polycations and/or polyanions present in the coacervate can covalently crosslink with the reinforcing component. In other aspects, the reinforcing component can occupy a space or “phase” in the coacervate, which ultimately increases the mechanical properties of the coacervate. Examples of reinforcing components useful herein are provided below. FIG. 4 shows the incorporation of water soluble or water-suspendable particles in the adhesive complex coacervate.

In one aspect, the reinforcing component is a polymerizable monomer. The polymerizable monomer entrapped in the complex coacervate can be any water soluble monomer capable of undergoing polymerization in order to produce an interpenetrating polymer network. In certain aspects, the interpenetrating network can possess nucleophilic groups (e.g., amino groups) that can react (i.e., crosslink) with the activated ester groups present on the polyanion. The selection of the polymerizable monomer can vary depending upon the application. Factors such as molecular weight can be altered to modify the solubility properties of the polymerizable monomer in water as well as the mechanical properties of the resulting coacervate,

The selection of the functional group on the polymerizable monomer determines the mode of polymerization. For example, the polymerizable monomer can be a polymerizable olefinic monomer that can undergo polymerization through mechanisms such as, for example, free radical polymerization and Michael addition reactions. In one aspect, the polymerizable monomer has two or more olefinic groups. In one aspect, the monomer comprises one or two actinically crosslinkable groups. The term “actinically crosslinkable group” in reference to curing or polymerizing means that the cros slinking between the polymerizable monomer is performed by actinic irradiation, such as, for example, UV irradiation, visible light irradiation, ionized radiation (e.g. gamma ray or X-ray irradiation), microwave irradiation, and the like. This can be performed in the presence of a photoinitiator, which is discussed in detail below. Actinic curing methods are well-known to a person skilled in the art. Examples of actinically crosslinkable group useful herein include, but are not limited to, a pendant acrylate group, methacrylate group, acrylamide group, methacrylamide group, allyl, vinyl group, vinylester group, or styrenyl group. Alternatively, polymerization can be performed in the presence of an initiator and coinitiator which are also discussed in detail below.

Examples of water-soluble polymerizable monomers include, but are not limited to, hydroxyalkyl methacrylate (HEMA), hydroxyalkyl acrylate, N-vinyl pyrrolidone, N-methyl-3-methylidene-pyrrolidone, allyl alcohol, N-vinyl alkylamide, N-vinyl-N-alkylamide, acrylamides, methacrylamide, (lower alkyl)acrylamides and methacrylamides, and hydroxyl-substituted (lower alkyl)acrylamides and -methacrylamides. In one aspect, the polymerizable monomer is a diacrylate compound or dimethacrylate compound. In another aspect, the polymerizable monomer is a polyalkylene oxide glycol diacrylate or dimethacrylate. For example, the polyalkylene can be a polymer of ethylene glycol, propylene glycol, or block co-polymers thereof. In one aspect, the polymerizable monomer is polyethylene glycol diacrylate or polyethylene glycol dimethacrylate. In one aspect, the polyethylene glycol diacrylate or polyethylene glycol dimethacrylate has a Mₙ of 200 to 2,000, 400 to 1,500, 500 to 1,000, 500 to 750, or 500 to 600.

In certain aspects, the interpenetrating polymer network is biodegradable and biocompatible for medical applications. Thus, the polymerizable monomer is selected such that a biodegradable and biocompatible interpenetrating polymer network is produced upon polymerization. For example, the polymerizable monomer can possess cleavable ester linkages. In one aspect, the polymerizable monomer is hydroxypropyl methacrylate (HPMA), which will produce a biocompatible interpenetrating network. In other aspects, biodegradable crosslinkers can be used to polymerize biocompatible water soluble monomers such as, for example, alkyl methacrylamides. The crosslinker could be enzymatically degradable, like a peptide, or chemically degradable by having an ester or disulfide linkage.

In another aspect, the reinforcing component can be a nanostructure. Depending upon the selection of the nanostructure, the polycation and/or polyanion can be covalently crosslinked to the nanostructure. Alternatively, the nanostructures can be physically entrapped within the coacervate. Nanostructures can include, for example, nanotubes, nanowires, nanorods, or a combination thereof. In the case of nanotubes, nanowires, and nanorods, one of the dimensions of the nanostructure is less than 100 nm

The nanostructures useful herein can be composed of organic and/or inorganic materials. In one aspect, the nanostructures can be composed of organic materials like carbon or inorganic materials including, but not limited to, boron, molybdenum, tungsten, silicon, titanium, copper, bismuth, tungsten carbide, aluminum oxide, titanium dioxide, molybdenum disulphide, silicon carbide, titanium diboride, boron nitride, dysprosium oxide, iron (III) oxide-hydroxide, iron oxide, manganese oxide, titanium dioxide, boron carbide, aluminum nitride, or any combination thereof.

In certain aspects, the nanostructures can be functionalized in order to react (i.e., crosslink) with the polycation and/or polyanion. For example, carbon nanotubes can be functionalized with amino groups or activated ester groups. In other aspects, it is desirable to use two or more different types of nanostructures. For example, a carbon nanostructure can be used in combination with one or more inorganic nanostructures.

In other aspects, the reinforcing component can be a water-insoluble filler. The filler can have a variety of different sizes and shapes, ranging from particles to fibrous materials. In one aspect, the filler is a nano-sized particle. Compared to micron-sized silica fillers, nanoscale fillers have several desirable properties. First, the higher specific surface area of nano- vs. microparticles increases the stress transfer from the polymer matrix to the rigid filler. Second, smaller volumes of nanofiller are required than of the larger micron-sized particles for a greater increase in toughness. Additionally, an important consequence of the smaller diameters and lower fill volumes of nanoparticles is reduced viscosity of the uncured adhesive, which has direct benefits for processability. This is advantageous, as the coacervate can retain its injectable character while potentially increasing bond strengths dramatically. Third, maximum toughening requires uniform dispersion of the filler particles within the coacervate. Nanoscale colloidal particles, again because of the small diameter, lend themselves more readily to stable dispersions within the coacervate.

In one aspect, the filler comprises a metal oxide, a ceramic particle, or a water insoluble inorganic salt. Examples of the nanoparticles or nanopowders useful herein include those manufactured by SkySpring Nanomaterials, Inc., which is listed below.

### Metals and Non-metal Elements

### Ag, 99.95%, 100 nm

### Ag, 99.95%, 20-30 nm

Ag, 99.95%, 20-30 nm, PVP coated

### Ag, 99.9%, 50-60 nm

Ag, 99.99%, 30-50 nm, oleic acid coated
Ag, 99.99%, 15 nm, 10 wt %, self-dispersible
Ag, 99.99%, 15 nm, 25 wt %, self-dispersible

### Al, 99.9%, 18 nm

### Al, 99.9%, 40-60 nm

### Al, 99.9%, 60-80 nm

Al, 99.9%, 40-60 nm, low oxygen

### Au, 99.9%, 100 nm

Au, 99.99%, 15 nm, 10 wt %, self-dispersible

### B, 99.9999%

### B, 99.999%

### B, 99.99%

### B, 99.9%

### B, 99.9%, 80 nm

### Diamond, 95%, 3-4 nm

### Diamond, 93%, 3-4 nm

### Diamond, 55-75%, 4-15 nm

### Graphite, 93%, 3-4 nm

### Super Activated Carbon, 100 nm

### Co, 99.8%, 25-30 nm

### Cr, 99.9%, 60-80 nm

### Cu, 99.5%, 300 nm

### Cu, 99.5%, 500 nm

### Cu, 99.9%, 25 nm

### Cu, 99.9%, 40-60 nm

### Cu, 99.9%, 60-80 nm

Cu, 5-7 nm, dispersion, oil soluble

### Fe, 99.9%, 20 nm

### Fe, 99.9%, 40-60 nm

### Fe, 99.9%, 60-80 nm

Carbonyl-Fe, micro-sized

### Mo, 99.9%, 60-80 nm

### Mo, 99.9%, 0.5-0.8 μm

Ni, 99.9%, 500 nm (adjustable)

### Ni, 99.9%, 20 nm

Ni coated with carbon, 99.9%, 20 nm

### Ni, 99.9%, 40-60 nm

### Ni, 99.9%, 60-80 nm

### Carbonyl-Ni, 2-3 μm

### Carbonyl-Ni, 4-7 μm

### Carbonyl-Ni—Al (Ni Shell, Al Core)

### Carbonyl-Ni—Fe Alloy

Pt, 99.95%, 5 nm, 10 wt %, self-dispersible

### Si, Cubic, 99%, 50 nm

Si, Polycrystalline, 99.99995%, lumps

### Sn, 99.9%, <100 nm

### Ta, 99.9%, 60-80 nm

### Ti, 99.9%, 40-60 nm

### Ti, 99.9%, 60-80 nm

### W, 99.9%, 40-60 nm

### W, 99.9%, 80-100 nm

### Zn, 99.9%, 40-60 nm

### Zn, 99.9%, 80-100 nm

### Metal Oxides

### AlOOH, 10-20 nm, 99.99%

Al₂O₃ alpha, 98+%, 40 nm
Al₂O₃ alpha, 99.999%, 0.5-10 μm
Al₂O₃ alpha, 99.99%, 50 nm
Al₂O₃ alpha, 99.99%, 0.3-0.8 μm
Al₂O₃ alpha, 99.99%, 0.8-1.5 μm
Al₂O₃ alpha, 99.99%, 1.5-3.5 μm
Al₂O₃ alpha, 99.99%, 3.5-15 μm
Al₂O₃ gamma, 99.9%, 5 nm
Al₂O₃ gamma, 99.99%, 20 nm
Al₂O₃ gamma, 99.99%, 0.4-1.5 μm
Al₂O₃ gamma, 99.99%, 3-10 μm
Al₂O₃ gamma, Extrudate
Al₂O₃ gamma, Extrudate

### Al(OH)₃, 99.99%, 30-100 nm

### Al(OH)₃, 99.99%, 2-10 μm

### Aluminium Iso-Propoxide (AIP), C₉H₂₁O₃Al, 99.9%

### AlN, 99%, 40 nm

### BaTiO3, 99.9%, 100 nm

### BBr₃, 99.9%

### B₂O₃, 99.5%, 80 nm

### BN, 99.99%, 3-4 μm

### BN, 99.9%, 3-4 μm

### B₄C, 99%, 50 nm

### Bi₂O₃, 99.9%, <200 nm

### CaCO₃, 97.5%, 15-40 nm

### CaCO₃, 15-40 nm

### Ca₃(PO₄)₂, 20-40 nm

### Ca₁₀(PO₄)₆(OH)₂, 98.5%, 40 nm

### CeO₂, 99.9%, 10-30 nm

### CoO, <100 nm

### Co₂O₃, <100 nm

### Co₃O₄, 50 nm

### CuO, 99+%, 40 nm

### Er₂O₃, 99.9%, 40-50 nm

Fe₂O₃ alpha, 99%, 20-40 nm
Fe₂O₃ gamma, 99%, 20-40 nm

### Fe₃O₄, 98+%, 20-30 nm

### Fe₃O₄, 98+%, 10-20 nm

### Gd₂O₃, 99.9%<100 nm

### HfO₂, 99.9%, 100 nm

### In₂O₃:SnO₂=90:10, 20-70 nm

### In₂O₃, 99.99%, 20-70 nm

### In(OH)₃, 99.99%, 20-70 nm

### LaB₆, 99.0%, 50-80 nm

### La₂O₃, 99.99%, 100 nm

### LiFePO₄, 40 nm

### MgO, 99.9%, 10-30 nm

### MgO, 99%, 20 nm

### MgO, 99.9%, 10-30 nm

### Mg(OH)₂, 99.8%, 50 nm

### Mn₂O₃, 98+%, 40-60 nm

### MoCl₅, 99.0%

### Nd₂O₃, 99.9%, <100 nm

### NiO, <100 nm

### Ni₂O₃, <100 nm

### Sb₂O₃, 99.9%, 150 nm

### SiO₂, 99.9%, 20-60 nm

SiO₂, 99%, 10-30 nm, treated with Silane Coupling Agents
SiO₂, 99%, 10-30 nm, treated with Hexamethyldisilazane
SiO₂, 99%, 10-30 nm, treated with Titanium Ester
SiO₂, 99%, 10-30 nm, treated with Silanes
SiO₂, 10-20 nm, modified with amino group, dispersible
SiO₂, 10-20 nm, modified with epoxy group, dispersible
SiO₂, 10-20 nm, modified with double bond, dispersible
SiO₂, 10-20 nm, surface modified with double layer, dispersible
SiO₂, 10-20 nm, surface modified, super-hydrophobic & oleophilic, dispersible
SiO₂, 99.8%, 5-15 nm, surface modified, hydrophobic & oleophilic, dispersible
SiO₂, 99.8%, 10-25 nm, surface modified, super-hydrophobic, dispersible
SiC, beta, 99%, 40 nm
SiC, beta, whisker, 99.9%
Si₃N₄, amorphous, 99%, 20 nm
Si₃N₄ alpha, 97.5-99%, fiber, 100 nm×800 nm

### SnO₂, 99.9%, 50-70 nm

### ATO, SnO₂:Sb₂O₃=90:10, 40 nm

TiO₂ anatase, 99.5%, 5-10 nm

### TiO₂ Rutile, 99.5%, 10-30 nm

TiO₂ Rutile, 99%, 20-40 nm, coated with SiO₂, highly hydrophobic
TiO₂ Rutile, 99%, 20-40 nm, coated with SiO₂/Al₂O₃
TiO₂ Rutile, 99%, 20-40 nm, coated with Al₂O₃, hydrophilic
TiO₂ Rutile, 99%, 20-40 nm, coated with SiO₂/Al₂O₃/Stearic Acid
TiO₂ Rutile, 99%, 20-40 nm, coated with Silicone Oil, hydrophobic

### TiC, 99%, 40 nm

### TiN, 97+%, 20 nm

### WO₃, 99.5%, <100 nm

### WS₂, 99.9%, 0.8 μm

### WCl₆, 99.0%

### Y₂O₃, 99.995%, 30-50 nm

### ZnO, 99.8%, 10-30 nm

ZnO, 99%, 10-30 nm, treated with silane coupling agents
ZnO, 99%, 10-30 nm, treated with stearic acid
ZnO, 99%, 10-30 nm, treated with silicone oil

### ZnO, 99.8%, 200 nm

### ZrO₂, 99.9%, 100 nm

### ZrO₂, 99.9%, 20-30 nm

### ZrO₂-3Y, 99.9%, 0.3-0.5 um

### ZrO₂-3Y, 25 nm

### ZrO₂-5Y, 20-30 nm

### ZrO₂-8Y, 99.9%, 0.3-0.5 μm

### ZrO₂-8Y, 20 nm

### ZrC, 97+%, 60 nm

In one aspect, the filler is nanosilica. Nanosilica is commercially available from multiple sources in a broad size range. For example, aqueous Nexsil colloidal silica is available in diameters from 6-85 nm from Nyacol Nanotechnologies, Inc. Amino-modified nanosilica is also commercially available, from Sigma Aldrich for example, but in a narrower range of diameters than unmodified silica. Nanosilica does not contribute to the opacity of the coacervate, which is an important attribute of the adhesives and glues produced therefrom.

In another aspect, the filler can be composed of calcium phosphate. In one aspect, the filler can be hydroxyapatite, which has the formula Ca₅(PO₄)₃OH. In another aspect, the filler can be a substituted hydroxyapatite. A substituted hydroxyapatite is hydroxyapatite with one or more atoms substituted with another atom. The substituted hydroxyapatite is depicted by the formula M₅X₃Y, where M is Ca, Mg, Na; X is PO₄ or CO₃; and Y is OH, F, Cl, or CO₃. Minor impurities in the hydroxyapatite structure may also be present from the following ions: Zn, Sr, Al, Pb, Ba. In another aspect, the calcium phosphate comprises a calcium orthophosphate. Examples of calcium orthophosphates include, but are not limited to, monocalcium phosphate anhydrate, monocalcium phosphate monohydrate, dicalcium phosphate dihydrate, dicalcium phosphate anhydrous, octacalcium phosphate, beta tricalcium phosphate, alpha tricalcium phosphate, super alpha tricalcium phosphate, tetracalcium phosphate, amorphous tricalcium phosphate, or any combination thereof. In other aspects, the calcium phosphate can also include calcium-deficient hydroxyapatite, which can preferentially adsorb bone matrix proteins.

In certain aspects, the filler can be functionalized with one or more amino or activated ester groups. In this aspect, the filler can be covalently attached to the polycation or polyanion. For example, aminated silica can be reacted with the polyanion possessing activated ester groups to form new covalent bonds.

In other aspects, the filler can be modified to produce charged groups such that the filler can form electrostatic bonds with the coacervates. For example, aminated silica can be added to a solution and the pH adjusted so that the amino groups are protonated and available for electrostatic bonding.

In one aspect, the reinforcing component can be micelles or liposomes. In general, the micelles and liposomes used in this aspect are different from the micelles or liposomes used as polycations and polyanions for preparing the coacervate. The micelles and liposomes can be prepared from the nonionic, cationic, or anionic surfactants described above. The charge of the micelles and liposomes can vary depending upon the selection of the polycation or polyanion as well as the intended use of the coacervate. In one aspect, the micelles and liposomes can be used to solubilize hydrophobic compounds such pharmaceutical compounds. Thus, in addition to be used as adhesives, the adhesive complex coacervates described herein can be effective as a bioactive delivery device.

### IV. Initiators and Other Components

In certain aspects, the coacervate also includes one or more initiators entrapped in the coacervate. Examples of initiators useful herein include a thermal initiator, a chemical initiator, or a photoinitiator. In one aspect, when the coacervate includes a polymerizable monomer as the reinforcing component, when the initiator is activated, polymerization of the polymerizable monomer entrapped in the coacervate occurs to produce the interpenetrating network. Additionally, crosslinking can occur between the polycation and polyanion as well as with the interpenetrating network.

Examples of photoinitiators include, but are not limited to a phosphine oxide, a peroxide group, an azide group, an α-hydroxyketone, or an α-aminoketone. In one aspect, the photoinitiator includes, but is not limited to, camphorquinone, benzoin methyl ether, 1-hydroxycyclohexylphenyl ketone, or Darocure® or Irgacure® types, for example Darocure® 1173 or Irgacure® 2959. The photoinitiators disclosed in European Patent No. 0632329, which are incorporated by reference, can be used herein. In other aspects, the photoinitiator is a water-soluble photoinitiator including, but not limited to, riboflavin, eosin, eosin y, and rose Bengal.

In one aspect, the initiator has a positively charged functional group. Examples include 2,2′-azobis[2-(5-methyl-2-imidazolin-2-yl)propane]-dihydrochloride; 2,2′-azobis[2-(2-imidazolin-2-yl)propane]dihydrochloride; 2,2′-azobis[2-(2-imidazo-lin-2-yl)propane]disulfate dehydrate; 2,2′-azobis(2-methylpropionamidine)dihydrochloride; 2,2′-azobis[2-(3,4,5,6-tetrahydropyrimidin-2-yl)propane]dihydrochloride; azobis {2-[1-(2-hydroxyethyl)-2-imidazolin-2-yl]propane}dihydrochloride; 2,2′-azobis(1-imino-1-pyrrolidino-2-ethylpropane)dihydrochloride and combinations thereof.

In another aspect, the initiator is an oil soluble initiator. In one aspect, the oil soluble initiator includes organic peroxides or azo compounds. Examples of organic peroxides include ketone peroxides, peroxyketals, hydroperoxides, dialkyl peroxides, diacyl peroxides, peroxydicarbonates, peroxyesters, and the like. Some specific non-limiting examples of organic peroxides that can be used as the oil soluble initiator include: lauroyl peroxide, 1,1-bis(t-hexylperoxy)-3,3,5-trimethylcyclohexane, 1,1-bis(t-butylperoxy)-3,3,5-trimethylcyclohexane, t-butylperoxylaurate, t-butylperoxyisopropylmonocarbonate, t-butylperoxy-2-ethylhexylcarbonate, di-t-butylperoxyhexahydro-terephthalate, dicumyl peroxide, 2,5-dimethyl-2,5-di(t-butylperoxy)hexane, di-t-butyl peroxide, t-butylperoxy-2-ethylhexanoate, bis(4-t-butylcyclohexyl)peroxydi-carbonate, t-amylperoxy-3,5,5-trimethylhexanoate, 1,1-di(t-amylperoxy)-3,3,5-trimethylcyclohexane, benzoyl-peroxide, t-butylperoxyacetate, and the like.

Some specific non-limiting examples of azo compounds that can be used as the oil soluble initiator include: 2,2′-azobis-isobutyronitrile, 2,2′-azobis-2,4-dimethylvaleronitrile, 1,1′-azobis-1-cyclohexane-carbonitrile, dimethyl-2,2′-azobisisobutyrate, 1,1′-azobis-(1-acetoxy-1-phenylethane), 4,4′-azobis(4-cyanopentanoic acid) and its soluble salts (e.g., sodium, potassium), and the like.

In one aspect, the initiator is a water-soluble initiator including, but not limited to, potassium persulfate, ammonium persulfate, sodium persulfate, and mixtures thereof. In another aspect, the initiator is an oxidation-reduction initiator such as the reaction product of the above-mentioned persulfates and reducing agents such as sodium metabisulfite and sodium bisulfite; and 4,4′-azobis(4-cyanopentanoic acid) and its soluble salts (e.g., sodium, potassium).

In certain aspects, multiple initiators can be used to broaden the absorption profile of the initiator system in order to increase the initiation rate. For example, two different photoinitiators can be employed that are activated by different wavelengths of light. In another aspect, a co-initiator can be used in combination with any of the initiators described herein. In one aspect, the co-initiator is 2-(diethylamino)ethyl acrylate, 2-(dimethylamino)ethyl acrylate, 2-(dimethylamino)ethyl benzoate, 2-(dimethylamino)ethyl methacrylate, 2-ethylhexyl 4-(dimethylamino)benzoate, 3-(dimethylamino)propyl acrylate, 4,4′-bis(diethylamino)benzophenone, or 4-(diethylamino)benzophenone.

In certain aspects, the initiator and/or co-initiator are covalently attached to the polycation and/or polyanion. For example, the initiator and/or co-initiator can be copolymerized with monomers used to make the polycation and/or polyanion. In one aspect, the initiators and co-initiators possess polymerizable olefinic groups such as acrylate and methacrylate groups (e.g., see examples of co-initiators above) that can be copolymerized with monomers described above used to make the polycation and polyanion. In another aspect, the initiators can be chemically grafted onto the backbone of the polycation and polyanion. Thus, in these aspects, the photoinitiator and/or co-initiator are covalently attached to the polymer and pendant to the polymer backbone. This approach will simply formulation and possibly enhance storage and stability.

The adhesive complex coacervates can optionally contain one or more multivalent cations (i.e., cations having a charge of +2 or greater). In one aspect, the multivalent cation can be a divalent cation composed of one or more alkaline earth metals. For example, the divalent cation can be a mixture of Ca⁺² and Mg⁺². In other aspects, transition metal ions with a charge of +2 or greater can be used as the multivalent cation. The concentration of the multivalent cations can determine the rate and extent of coacervate formation. Not wishing to be bound by theory, weak cohesive forces between particles in the fluid may be mediated by multivalent cations bridging excess negative surface charges. The amount of multivalent cation used herein can vary. In one aspect, the amount is based upon the number of anionic groups and cationic groups present in the polyanion and polycation.

### V. Preparation of Adhesive Complex Coacervates

The synthesis of the adhesive complex coacervates described herein can be performed using a number of techniques and procedures. Exemplary techniques for producing the coacervates are provided in the Examples. In one aspect, an aqueous solution of polycation is mixed with an aqueous solution of polyanion, where one or both of the solutions contain optionally contain one or more reinforcing components (e.g., polymerizable monomers, fillers, initiators, etc.). In certain aspects, the pH of each solution can be adjusted to a desired pH (e.g., physiological pH) prior to mixing with one another to produce the complex coacervate. Alternatively, after mixing the polycation, polyanion, polymerizable monomer, and optional components, the pH of the resulting solution can be adjusted to produce the complex coacervate. Upon mixing, the adhesive complex coacervate forms a fluid that settles to the bottom of the solution, at which time the supernatant is removed and the complex coacervate is ready for use to produce the adhesive.

After the adhesive complex coacervate is formed, it is subsequently cured to induce crosslinking within the coacervate to produce a cured adhesive complex coacervate. The cured adhesive complex coacervate is also referred to herein as “an adhesive.” Depending upon the selection of starting materials, varying degrees of crosslinking can occur throughout the coacervate during curing. In one aspect, the polycations and polyanions can be crosslinked with one another by covalent bonds upon curing.

In one aspect, after the adhesive complex coacervate has been produced and applied to a substrate or adherend it can be converted to a load bearing adhesive bond using techniques known in the art. In one aspect, the adhesive can be produced by the process comprising

(a) providing an adhesive complex coacervate comprising (i) at least one polyanion comprising a plurality of carboxyl groups, and (ii) at least one polycation comprising a plurality of nucleophilic groups that can react with the activated ester groups to produce a new covalent bond between the polycation and the polyanion; and
(b) contacting the polyanion with a reagent to convert at least one carboxyl group to an activated ester, wherein a nucleophilic group present on the polycation reacts with the activated ester to produce a new covalent bond.

In this aspect, step (b) involves curing the adhesive complex coacervate in order to crosslink the polycation and polyanion. In one aspect, after the complex coacervates has been prepared and applied to an adherend, the coacervate is contacted with a reagent that converts the carboxyl groups present on the polyanion to activated ester groups. Upon formation of the activated ester groups, the nucleophilic groups present on the polycation react with the activated ester groups to form covalent bonds between the polyanion and polycation and cure the coacervate.

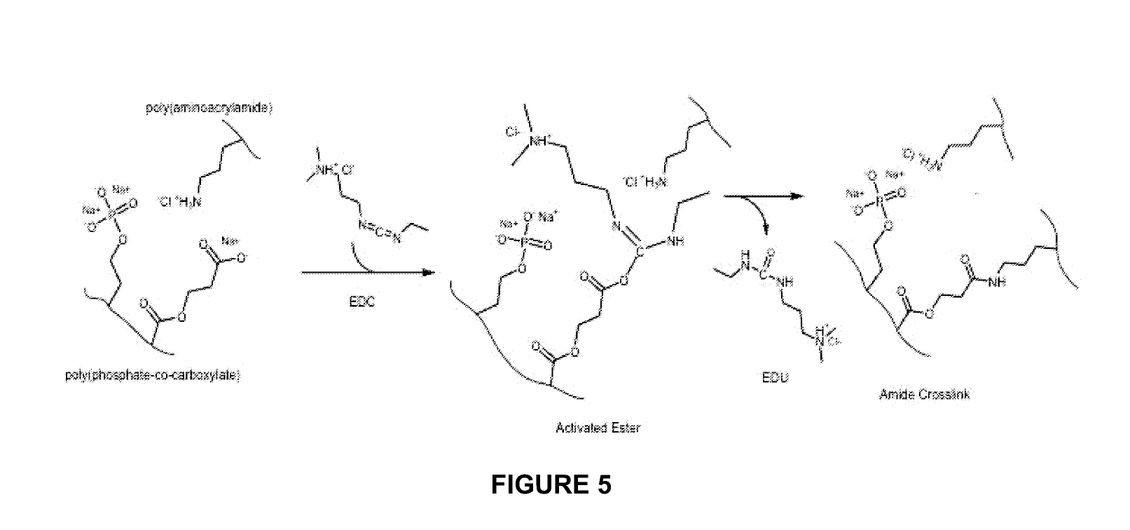

Any reagent typically used in organic synthesis for producing activated esters can be used herein. In one aspect, the reagent is a carbodiimide such as, for example, ethylenediamine carbodiimide (EDC). In other aspects, the reagent can be an N-hydroxysuccinimide, a nitrophenol, or a fluorophenol (e.g., pentafluorophenol). An exemplary procedure for crosslinking (i.e., curing) the polycation and polyanion is provided in FIG. 5 and the Examples. In the case of EDC, it is converted to a nontoxic urea species known as 1-ethyl-3-(3-dimethylaminopropyl)urea (EDU) (see FIG. 5 and Examples). This is desirable when the coacervates described herein are used in biomedical applications.

In the aspects above, the time and degree of curing can be controlled by the addition of the reagent used to produce the activated ester groups on the polyanion. In other aspects, the polyanion can possess activated ester groups prior to forming the coacervate with the polycation and subsequent curing. In one aspect, the polyanion with free carboxyl groups can be reacted with any of the reagents described above to convert the carboxyl groups to activated esters. In the alternative, monomers containing an activated ester group can be polymerized with other monomers to produce the polyanion. In these aspects, the polycation with activated ester groups can crosslink rapidly with the polycation.

In other aspects, when a polymerizable monomer (i.e., a reinforcing component) is present in the coacervates, the polycations and/or polyanions can be crosslinked with the interpenetrating network. For example, the polymerizable monomer can possess groups that can covalently crosslink with the polycation and/or polyanion, which enhances the overall mechanical properties of the coacervate.

The method of polymerizing the polymerizable monomer to produce the interpenetrating network can vary depending upon the nature of the polymerizable monomer. For example, if the polymerizable monomer has one or more polymerizable olefinic groups, an initiator and a co-initiator can be incorporated into the coacervate using the methods described above, and the coacervate can be exposed to light. Here, the polymerizable monomer polymerizes in the coacervate to produce the interpenetrating network. Any of the initiators and co-initiators described above can be used herein.

In certain aspects, the polycation and/or polyanion can be covalently attached to the interpenetrating network. For example, the polycation and polyanion can possess nucleophilic groups (e.g., thiols or amines) capable of reacting with groups on the interpenetrating network (e.g., olefinic groups).

In other aspects, when the reinforcing component is a filler, the filler can be functionalized such that it can form covalent or non-covalent bonds with the polycation, polyanion, and, in certain aspects, the interpenetrating network. For example, if the filler is functionalized with olefinic groups such as acrylate groups, it can polymerize with the polymerizable monomer such that the filler is covalently bonded to the resulting interpenetrating network. Alternatively, the filler can be modified with nucleophilic groups capable of reacting with electrophilic groups on the polycation and/or polyanion. In other aspects, the filler can possess groups that permit electrostatic interactions between the polycation and/or polyanion.

In other aspects, when the reinforcing component does not possess groups capable of forming a covalent bond with the coacervate, the reinforcing component can enhance the mechanical properties of the coacervate by occupying or filling gaps in the coacervate. In this aspect, the reinforcing component is physically entrapped within the coacervate. The reinforcing component forms a rigid internal skeleton, which enhances the mechanical properties of the coacervate,

The adhesive complex coacervates described herein have several desirable features when compared to conventional adhesives. The adhesive complex coacervates described herein can be delivered underwater without dispersing into the water because they are phase separated from water although being water-borne, they have low interfacial tension with water and wettable substrates; when applied to a wet substrate they spread over the interface rather than beading up. The adhesive complex coacervates are effective in bonding two adherends together, particularly when the adherends are wet or will be exposed to an aqueous environment. The crosslinking between the polycation and polyanion enhances the mechanical properties of the coacervate including, but not limited to, cohesion (i.e., internal strength), fracture toughness, extensibility, fatigue resistance, elastic modulus, the ability to release and bioactive agents, dimensional stability after curing, etc.

### VI. Kits

The polycations and polyanions described herein can be stored as dry powders for extended periods of time. This feature is very useful for preparing the coacervates and ultimately the adhesives when desired. Thus, described herein are kits for making the complex coacervates and adhesives described herein. In one aspect, the kit comprises (1) at least one polyanion comprising at least one carboxyl group; (2) at least one polycation comprising a plurality of nucleophilic groups that can react with the activated ester groups to produce a new covalent bond between the polycation and the polyanion; and (3) a reagent to convert at least one carboxyl group on the polyanion to an activated ester. In another aspect, the kit comprises (1) at least one polyanion comprising at least one carboxyl group; (2) at least one polycation comprising a plurality of nucleophilic groups that can react with the activated ester groups to produce a new covalent bond between the polycation and the polyanion; (3) a reagent to convert at least one carboxyl group on the polyanion to an activated ester. and (4) a reinforcing component. In a further aspect, the kit comprises (1) at least one polyanion comprising at least one carboxyl group; (2) at least one polycation comprising a plurality of nucleophilic groups that can react with the activated ester groups to produce a new covalent bond between the polycation and the polyanion; (3) a reagent to convert at least one carboxyl group on the polyanion to an activated ester; (4) a reinforcing component, and (5) an initiator and optional coinitiator.

In another aspect, the kit includes (1) at least one polyanion comprising a plurality of activated ester groups, and (2) at least one polycation comprising a plurality of nucleophilic groups that can react with the activated ester groups to produce a new covalent bond between the polycation and the polyanion.

When stored as dried powders, water can be added to the polycation and/or polyanion to produce the coacervate. In one aspect, prior to lyophilizing the polycation and polyanion in order to produce a dry powder, the pH of the polycation and polyanion can be adjusted such that when they are admixed in water the desired pH is produced without the addition of acid or base. For example, excess base can be present in the polycation powder which upon addition of water adjusts the pH accordingly.

### VII. Applications of the Adhesive Complex Coacervates

The adhesive complex coacervates and adhesives described herein have numerous benefits with respect to their use as biological glues and delivery devices. For example, the coacervates have low initial viscosity, specific gravity greater than one, and containing a significant fraction of water by weight, low interfacial tension in an aqueous environment, all of which contribute to their ability to adhere to a wet surface. They are water-borne eliminating the need for potentially toxic solvents. Despite being water-borne they are phase separated from water. This allows the adhesives complex coacervate to be delivered underwater without dispersing. The adhesive complex coacervates are dimensional stable after crosslinking so that when applied in a wet (e.g., physiological) environment they do not swell. The lack of swelling, i.e., absorption of water, is due to the phase-separated nature of the copolymer network. This is of critical importance for medical adhesives; swelling after application can cause damage to surrounding tissues and pain. Dimensional stability is a major advantage over tissue adhesives/sealants based on crosslinked PEG hydrogels. An additional advantage with respect to the bonding mechanism (i.e., crosslinking) of the adhesive complex coacervates includes low heat production during setting, which prevents damage to living tissue.

One approach for applying the adhesive complex coacervate to the substrate involves the use of a multi-compartment syringe. In one aspect, a double-compartment or barrel syringe can be used. Thus, in this aspect, the adhesive complex coacervate can be applied at distinct and specific regions of the substrate. In one aspect, one barrel of the syringe can contain a coacervate composed of polyanion with a plurality of free carboxyl groups and polycation, and the second barrel contains reagent for converting the free carboxyl groups to activated esters.

The properties of the adhesive complex coacervates described herein make them ideal for underwater applications such as the administration to a subject. For example, the adhesive complex coacervates and adhesives produced therefrom can be used to repair a number of different bone fractures and breaks. The coacervates adhere to bone (and other minerals) through several mechanisms. The surface of the bone's hydroxyapatite mineral phase (Ca₅(PO₄)₃(OH)) is an array of both positive and negative charges. The negative groups present on the polyanion (e.g., phosphate groups) can interact directly with the positive surface charges or it can be bridged to the negative surface charges through the cationic groups on the polycation and/or multivalent cations. Likewise, direct interaction of the polycation with the negative surface charges would contribute to adhesion. Alternatively, oxidized crosslinkers can couple to nucleophilic sidechains of bone matrix proteins.

Examples of such breaks include a complete fracture, an incomplete fracture, a linear fracture, a transverse fracture, an oblique fracture, a compression fracture, a spiral fracture, a comminuted fracture, a compacted fracture, or an open fracture. In one aspect, the fracture is an intra-articular fracture or a craniofacial bone fracture. Fractures such as intra-articular fractures are bony injuries that extend into and fragment the cartilage surface. The adhesive complex coacervates and adhesives may aid in the maintenance of the reduction of such fractures, allow less invasive surgery, reduce operating room time, reduce costs, and provide a better outcome by reducing the risk of post-traumatic arthritis.

In other aspects, the adhesive complex coacervates and adhesives produced therefrom can be used to join small fragments of highly comminuted fractures. In this aspect, small pieces of fractured bone can be adhered to an existing bone. It is especially challenging to maintain reduction of the small fragments by drilling them with mechanical fixators. The smaller and greater number of fragments the greater the problem. In one aspect, the adhesive complex coacervate may be injected in small volumes to create spot welds as described above in order to fix the fracture rather than filling the entire crack followed by curing the adhesive complex coacervate. The small biocompatible spot welds would minimize interference with healing of the surrounding tissue and would not necessarily have to be biodegradable. In this respect it would be similar to permanently implanted hardware.

In other aspects, the adhesive complex coacervates and adhesives produced therefrom can be used to secure a patch to bone and other tissues such as, for example, cartilage, ligaments, tendons, soft tissues, organs, and synthetic derivatives of these materials. In one aspect, the patch can be a tissue scaffold or other synthetic materials or substrates typically used in wound healing applications. Using the complexes and spot welding techniques described herein, the adhesive complex coacervates and adhesives produced therefrom can be used to position biological scaffolds in a subject. Small adhesive tacks composed of the adhesive complex coacervates described herein would not interfere with migration of cells or transport of small molecules into or out of the scaffold. In certain aspects, the scaffold can contain one or more drugs that facilitate growth or repair of the bone and tissue. In other aspects, the scaffold can include drugs that prevent infection such as, for example, antibiotics. For example, the scaffold can be coated with the drug or, in the alternative, the drug can be incorporated within the scaffold so that the drug elutes from the scaffold over time.

The adhesive complex coacervates and adhesives produced therefrom have numerous dental applications. For example, the adhesive complex coacervates can be used to seal breaks or cracks in teeth, for securing crowns, or allografts, or seating implants and dentures. The adhesive complex coacervate can be applied to a specific points in the mouth (e.g., jaw, sections of a tooth) followed by attaching the implant to the substrate and subsequent curing.

In other aspects, the adhesive complex coacervates and adhesives produced therefrom can adhere a substrate to bone. For example, implants made from titanium oxide, stainless steel, or other metals are commonly used to repair fractured bones. The adhesive complex coacervate can be applied to the metal substrate, the bone, or both prior to adhering the substrate to the bone. In other aspects, the substrate can be a fabric (e.g., an internal bandage), a tissue graft, or a wound healing material. Thus, in addition to bonding bone fragments, the adhesive complex coacervates described herein can facilitate the bonding of substrates to bone, which can facilitate bone repair and recovery.

It is also contemplated that the adhesive complex coacervates and adhesives produced therefrom can encapsulate one or more bioactive agents. The bioactive agents can be any drug including, but not limited to, antibiotics, pain relievers, immune modulators, growth factors, enzyme inhibitors, hormones, mediators, messenger molecules, cell signaling molecules, receptor agonists, or receptor antagonists.

In another aspect, the bioactive agent can be a nucleic acid. The nucleic acid can be an oligonucleotide, deoxyribonucleic acid (DNA), ribonucleic acid (RNA), or peptide nucleic acid (PNA). The nucleic acid of interest can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In other aspects, the bioactive agent is used in bone treatment applications. For example, the bioactive agent can be bone morphogenetic proteins (BMPs) and prostaglandins. When the bioactive agent is used to treat osteoporosis, bioactive agents known in the art such as, for example, bisphonates, can be delivered locally to the subject by the adhesive complex coacervates and adhesives described herein.

In certain aspects, the filler used to produce the coacervate can also possess bioactive properties. For example, when the filler is a silver particle, the particle can also behave as an anti-bacterial agent. The rate of release can be controlled by the selection of the materials used to prepare the complex as well as the charge of the bioactive agent if the agent is a salt. Thus, in this aspect, the insoluble solid can perform as a localized controlled drug release depot. It may be possible to simultaneously fix tissue and bones as well as deliver bioactive agents to provide greater patient comfort, accelerate bone healing, and/or prevent infections.

The adhesive complex coacervates and adhesives produced there from can be used in a variety of other surgical procedures. For example, adhesive complex coacervates and adhesives produced therefrom can be used to treat ocular wounds caused by trauma or by the surgical procedures. In one aspect, the adhesive complex coacervates and adhesives produced therefrom can be used to repair a corneal or schleral laceration in a subject. In other aspects, adhesive complex coacervates can be used to facilitate healing of ocular tissue damaged from a surgical procedure (e.g., glaucoma surgery or a corneal transplant). The methods disclosed in U.S. Published Application No. 2007/0196454, which are incorporated by reference, can be used to apply the coacervates described herein to different regions of the eye.

In other aspects, the adhesive complex coacervates and adhesives produced therefrom can be used to inhibit blood flow in a blood vessel of a subject (i.e., embolic applications). In general, the adhesive complex coacervate is injected into the vessel followed by polymerizing the polymerizable monomer as described above to partially or completely block the vessel. This method has numerous applications including hemostasis or the creation of an artificial embolism to inhibit blood flow to a tumor or aneurysm or other vascular defect.

The adhesive complex coacervates described herein to seal the junction between skin and an inserted medical device such as catheters, electrode leads, needles, cannulae, osseo-integrated prosthetics, and the like. In this aspect, the coacervates prevent infection at the entry site when the device is inserted in the subject. In other aspects, the coacervates can be applied to the entry site of the skin after the device has been removed in order to expedite wound healing and prevent further infection.

In another aspect, the adhesive complex coacervates described herein can be used to close or seal a puncture in an internal tissue or membrane. In certain medical applications, internal tissues or membranes are punctured, which subsequently have to be sealed in order to avoid additional complications. Alternatively, the adhesive complex coacervates described herein can be used to adhere a scaffold or patch to the tissue or membrane in order to prevent further damage and facilitate wound healing.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Coacervate Formation

The polycation used to produce the coacervates is depicted in FIG. 2. The polycation is four-arm polyaminoacrylamide synthesized by RAFT. RAFT provides controlled Mₙ and copolymer structure. The branching RAFT has a cluster of hydrolysable ester bonds in the center to promote degradation of the polymer and the adhesive. The polyanion is a polyphospho-co-carboxylate (25.5% mol methacrylic acid, 55.9% MOEP, 17.5% HEMA).

The coacervates were prepared by mixing the tetra-polyamineacrylamide (17.7 mol % amine) with polyphospho-co-carboxylate. The amine to phosphate ratio was fixed at 0.8, and calcium was used as a divalent cation at a ratio of 0.6 to phosphate. All coacervates were formed in a 150 mM NaCl solution and the pH adjusted to 7.4. EDC ratio was based on the molar ratio of EDC to carboxylate ion, and was added immediately prior to crosslinking at a concentration of 1 mg/l μL. The molar ratio of amines to carboxylates was 1.7:1.

### Characterization of the Coacervates

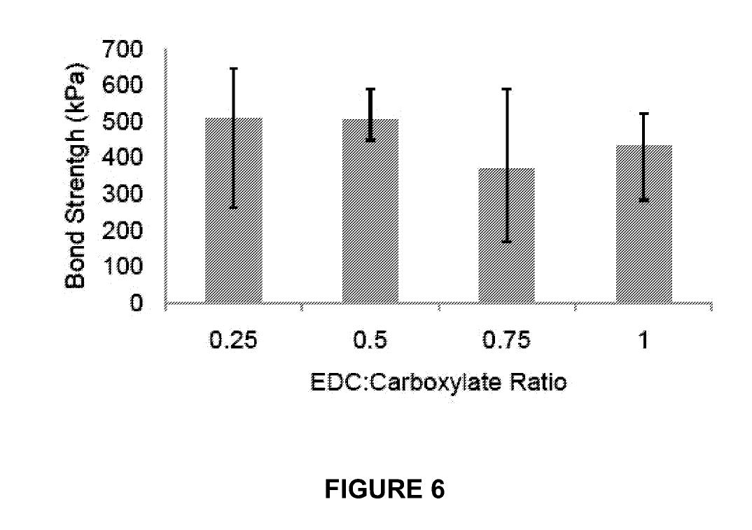

Bond strength measurements were done using an Instron 3342 using a tensile lap shear configuration (0.0200 mm/sec). Aluminum strips and pig skin tissue on aluminum were prepared according to ASTM standards. All samples where allowed to crosslink in a 150 mM NaCl solution at 37° C. before being tested. The results are shown in FIG. 6.

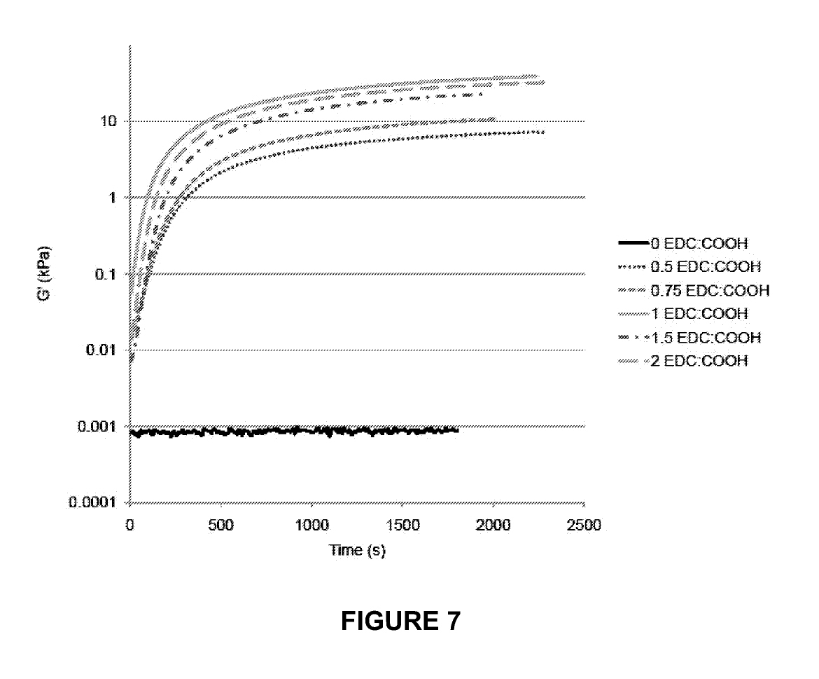

The rheological time sweep measurements were taken using a TA Instruments rheometer, AR 2000 EX, with a cone-plate geometry (diameter 20 mm, angle 4°). The temperature was maintained at 37° C. using a Peltier plate. For each sample, the frequency was set at 1 Hz and strain of 1.0%, which were both in the linear regime. FIG. 7 shows the time sweep measurements for different coacervates with varying EDC/carboxylate ratios.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1-64. (canceled)

65. A cured adhesive complex coacervate produced by the process comprising reacting (a) at least one polyanion comprising a plurality of activated ester groups, and (b) at least one polycation comprising a dendrimer comprising a plurality of amino groups, wherein the amino groups react with the activated ester groups to produce a new covalent bond between the polycation and the polyanion.

66. The adhesive complex coacervate of claim 65, wherein the dendrimer has 3 to 20 arms, wherein each arm comprises an amino group.

67. The adhesive complex coacervate of claim 65, wherein the polyanion comprises (1) two or more sulfate, sulfonate, borate, boronate, phosphonate, or phosphate groups and (2) a plurality of activated ester groups.

68. The adhesive complex coacervate of claim 65, wherein the polyanion comprises a polyphosphate and a plurality of activated ester groups.

69. The adhesive complex coacervate of claim 68, wherein the polyphosphate comprises a polyacrylate comprising one or more pendant phosphate groups.

70. The adhesive complex coacervate of claim 65, wherein the polyanion comprises a polymer comprising at least one fragment comprising the formula X 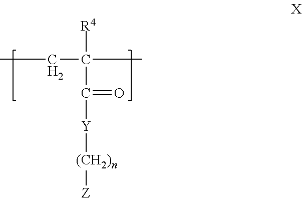
wherein R⁴ is hydrogen or an alkyl group;
n is from 1 to 10;
Y is oxygen, sulfur, or NR³⁰, wherein R³⁰ is hydrogen, an alkyl group, or an aryl group;
Z is an activated ester,
or the pharmaceutically-acceptable salt thereof.

71. The adhesive complex coacervate of claim 70, wherein the polyanion further comprises at least one fragment comprising the formula II 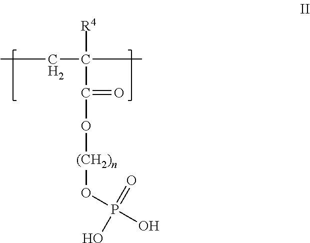
wherein R⁴ is hydrogen or an alkyl group, and n is from 1 to 10, or the pharmaceutically-acceptable salt thereof.

72. The adhesive complex coacervate of claim 65, wherein the coacervates further comprises a reinforcing component.

73. The adhesive complex coacervate of claim 65, wherein the coacervate further comprises at least one multivalent cation.

74. The adhesive complex coacervate of claim 73, wherein the multivalent cation comprises one or more divalent cations.

75. The adhesive complex coacervate of claim 65, wherein the coacervate further comprises one or more bioactive agents encapsulated in the coacervate.

76. A cured adhesive complex coacervate produced by the process comprising
(a) providing an adhesive complex coacervate comprising (i) at least one polyanion comprising a plurality of carboxyl groups, and (ii) at least one polycation comprising a dendrimer comprising a plurality of amino groups that can react with the activated ester groups to produce a new covalent bond between the polycation and the polyanion; and
(b) contacting the polyanion with a reagent to convert at least one carboxyl group to an activated ester, wherein an amino group present on the polycation reacts with the activated ester to produce a new covalent bond.

77. A kit comprising (1) at least one polyanion comprising a plurality of activated ester groups, and (2) at least one polycation comprising a dendrimer comprising a plurality of amino groups that can react with the activated ester groups to produce a new covalent bond between the polycation and the polyanion.

78. An adhesive complex coacervate comprising (a) at least one polyanion comprising a plurality of carboxyl groups, and (b) at least one polycation comprising a dendrimer comprising a plurality of amino groups that can react with the activated ester groups to produce a new covalent bond between the polycation and the polyanion.

79. A method for repairing a bone fracture in a subject, comprising (1) contacting the fractured bone with the adhesive complex coacervate of claim 78 and (2) curing the adhesive complex coacervate.

80. A method for adhering a substrate to a bone of a subject comprising (1) contacting the bone with the adhesive complex coacervate of claim 78; (2) applying the substrate to the coated bone; and (3) curing the adhesive complex coacervate.

81. A method for adhering a bone-tissue scaffold to a bone of a subject comprising (1) contacting the bone and tissue with the adhesive complex coacervate of claim 78; (2) applying the bone-tissue scaffold to the bone and tissue; and (3) curing the adhesive complex coacervate.

82. A method to bond a soft tissue scaffold to soft tissue or two soft tissue scaffolds or two soft tissues comprising (1) applying the adhesive complex coacervate of claim 78 to the tissue scaffold and/or soft tissue; and (2) curing the adhesive complex coacervate.

83. A method for treating an ocular wound comprising (1) applying to the wound the adhesive complex coacervate of claims 78; and (2) curing the adhesive complex coacervate.

84. A method of closing or sealing a puncture in internal tissue or membrane comprising (1) adhering a patch to the puncture with the adhesive complex coacervate of claims 78; and (2) curing the adhesive complex coacervate.

85. A method for inhibiting blood flow in a blood vessel of a subject comprising (1) introducing the adhesive complex coacervate of claim 78 into the vessel; and (2) optionally curing the adhesive complex coacervate.

86. A method for delivering one or more bioactive agents to a subject comprising administering the cured adhesive complex coacervate of claim 65 to the subject, wherein the bioactive agent is encapsulated in the cured adhesive complex coacervate.

87. A method for delivering one or more bioactive agents to a subject comprising administering the adhesive complex coacervate of claim 78 to the subject, wherein the bioactive agent is encapsulated in the adhesive complex coacervate.
